(19) United States
(12) Patent Application Publication
(10) Pub. No.: US 2008/0033274 A1
(43) Pub. Date: Feb. 7, 2008

(54) Device and Method for Monitoring the Functioning of the Lower Esophageal Sphincter

(75) Inventor: Stefan Postius, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/596,805

(22) PCT Filed: May 23, 2005
(86) PCT No.: PCT/EP05/52345

§ 371 Date: Jan. 24, 2007

(30) Foreign Application Priority Data
May 25, 2004 (EP) 04102304.5
Nov. 25, 2004 (EP) 04106097.1

(51) Int. Cl. A61B 5/00

(52) U.S. Cl. 600/350

(57) ABSTRACT

The present invention relates to a device for monitoring the functioning of the lower esophageal sphincter in mammals.

### TECHNICAL FIELD

The present invention relates to a device and a method for monitoring the functioning of the lower esophageal sphincter in mammals. The device and the method can be used inter alia in the diagnosis of gastroesophageal reflux disease.

### BACKGROUND ART

The gastrointestinal (GI) tract extends from the mouth to the anus, and includes the esophagus, stomach, small and large intestines, and rectum. Along the way, ring-like muscle fibers called sphincters control the passage of food from one specialized portion of the GI tract to another. The GI tract is lined with a mucosal layer about 1-2 mm thick that absorbs and secretes substances involved in the digestion of food and protects the body's own tissue from self-digestion. The esophagus is a muscular tube that extends from the pharynx through the esophageal hiatus of the diaphragm to the stomach. Peristalsis of the esophagus propels food toward the stomach as well as clears any refluxed contents of the stomach.

The junction of the esophagus with the stomach is controlled by the lower esophageal sphincter (LES), a thickened circular ring of smooth esophageal muscle. The LES straddles the squamocolumnar junction, or z-line—a transition in esophageal tissue structure that can be identified endoscopically. At rest, the LES maintains a high-pressure zone between 10 and 30 mm Hg above intragastric pressures. The LES relaxes upon arrival of a peristaltic wave, and allows food to pass through to the stomach. After food passes into the stomach, the LES constricts to prevent the contents from regurgitating into the esophagus. The resting tone of the LES is maintained by muscular and nerve mechanisms, as well as different reflex mechanisms and physiologic alterations. Transient lower esophageal sphincter relaxations (TLESR) manifest independently of swallowing. These TLESR are often associated with transient gastroesophageal reflux in healthy people, for example if gas developed in the stomach is to be released due to different gas pressures into the esophagus and further to the mouth or nose, for example in form of belches. Muscular contractions of the diaphragm around the esophageal hiatus during breathing serve as a diaphragmatic sphincter that offers additional augmentation of lower esophageal sphincter pressure to prevent reflux.

A variety of diseases and ailments arising from the dysfunction of the lower esophageal sphincter, typically manifest through transient lower esophageal sphincter relaxations (TLESR), which lead to reflux of stomach acids into the esophagus. This condition, the so called Gastroesophageal Reflux Disease (GERD), creates discomfort which symptoms include, but are not limited to, heartburn and/or acid regurgitation. The reflux of stomach acid can begin to erode the lining of the esophagus—a condition that can progress to esophagitis and a pre-cancerous condition known as Barrett's Epithelium. Complications of the disease can progress to difficulty and pain in swallowing, stricture, perforation and bleeding, anemia, and weight loss. Dysfunction of the diaphragmatic sphincter, such as that caused by a hiatal hemia, can aggravate the problem of LES relaxations.

GERD is a common and highly prevalent condition characterized by the typical symptoms such as for example heartburn, acid regurgitation, and pain on swallowing. Other associated reflux symptoms are for example. burning in the throat, dysphagia, pressure in the upper and lower abdomen, sore throat, nausea or vomiting. The term GERD is generally understood to comprise both, erosive esophagitis and endoscopy negative reflux disease (enGERD). GERD affects all individuals who are exposed to the risk of physical complications from gastro-esophageal reflux or who experience clinically significant impairment of health-related well being (quality of life) due to reflux-related symptoms, after adequate reassurance of the benign nature of their symptoms.

Due to the aforementioned implications, the diagnosis of GERD at an early stage is of utmost importance and therefore the accurate diagnosis of dysfunctions of the LES can support the physician in the choice of the required therapy. For the diagnosis of dysfuncitons of the LES several methods are known to the person skilled in the art some of which are used in daily practice by physicians already.

One known method for the diagnosis of dysfunctions of the LES is a method using a pH probe which is known for example from Sarani B, Gleiber M, Evans S R Esophageal pH monitoring, indications, and methods.: J Clin Gastroenterol. 2002 March; 34(3):200-6. The pH probe is introduced orally or intranasally and pushed forward until the pH indicates the gastric acidity. Thereupon the probe is retracted for a distance of 5 cm. One prerequisite for using a pH probe in the measurement of dysfunctions of the LES is that the detection of reflux into the esophagus is restricted to reflux with a low pH value. Therefore the use of that method is restricted to a certain population, like for example individuals which are not subjected to a treatment with pharmaceuticals which inhibit acid secretion, such as for example H₂ blockers (e.g. cimetidine, ranitidine) or H⁺/K⁺-ATPase inhibitors (e.g. omeprazole, esomeprazole, lansoprazole, pantoprazole or rabeprazole) or acid pump antagonists.

Another known method for the diagnosis of dysfunctions of the LES is the detection of esophageal reflux events by use of multichannel intraluminal impedance measurements (see for example Biomed Sci Instrum. 2001; 37:55-61) Richter J E. Diagnostic tests for gastroesophageal reflux disease. Am J Med Sci. 2003 November; 326(5):300-8. As in the case of the pH-electrode, the probe is advanced orally or intranasally down to the LES. The probe registrates ascending gastric liquid irrespectively of its pH. This technique mainly measures regurgitation of gastric liquid. Very recently, the technique has been shown to detect gaseous reflux as well. In order to differentiate whether the gas is coming from the stomach or coming down with a swallow, the technique depends on several electrodes to identify the direction of the gas flux.

Still another known method for the diagnosis of dysfunctions of the LES is based on a micro-manometrie technique usually combined with the so called Dentsleeve technique (Richter J E. Diagnostic tests for gastroesophageal reflux disease. Am J Med Sci. 2003 November; 326(5):300-8 and Fermin Mearin and Juan-R. Malagelada. Complete lower esophageal sphincter relaxation observed in some achalasia patients is functionally inadequate Am. J. Physiol. Gastrointest. Liver Physiol. 278: G376-G383, 2000). Again, this probe is advanced orally or intranasally down to the LES. The assembly consists of different tiny tubes, glued together to yield a multilumen tube. The different tubes end at distinct places of the mulblumen tube. Upon positioning in the esophagus, the tubes are perfused with water and the changes in resistance due to peristaltic waves are continuously recorded. The Dentsleeve is positioned in the LES and results in continuous measurement of the pressure in the LES.

The U.S. Pat. No. 3,334,623 describes an electrochemical sensor having a membrane covering the sensing electrode which is mounted in a catheter for in vivo analysis of body fluids.

The U.S. Pat. No. 5,117,827 describes a combination probe including a flexible tube having a pH probe and a pressure sensor, a use of the same and a method for monitoring gastric acid reflux in a patient.

The US patent application US 2004/0138586 describes an apparatus including a catheter for insertion into the stomach to introduce air into the stomach through the catheter and a pressure measurement device to sense a pressure in the stomach. The apparatus is used for measuring gastric yield pressure and distal esophageal compliance.

The international patent application WO 90/01894 describes a tonometric combination apparatus for measuring a fluid of interest of the condition of an internal organ of a human or other mammal in vivo.

### DISCLOSURE OF THE INVENTION

Technical Problem

The problem of the present invention is to provide a device and a method for the diagnosis of dysfunctions of the LES which avoid the disadvantages and restrictions of the methods mentioned above.

Technical Solution

The problem is solved by a device and a method for monitoring the functioning of the lower esophageal sphincter in mammals wherein the device comprises a sensor for a marker gas.

In one aspect (aspect A) the invention relates to a device for monitoring the functioning of the lower esophageal sphincter in mammals which comprises a sensor for a marker gas.

The invention further relates to a device for monitoring the functioning of the lower esophageal sphincter in mammals which comprises a measuring probe, a sensor for a marker gas and a device which records the amount of marker gas detected in the sensor dependent of time.

The invention further relates to a device for monitoring the functioning of the lower esophageal sphincter in mammals which comprises a measuring probe, a sensor for a marker gas and a device which records the amount of marker gas detected in the sensor dependent of time, wherein the measuring probe comprises an open measuring probe head.

The invention further relates to a device for monitoring the functioning of the lower esophageal sphincter in mammals which comprises a measuring probe, a sensor for a marker gas and a device which records the amount of marker gas detected in the sensor dependent of time, wherein the measuring probe comprises a closed measuring probe head.

In a preferred device according to the invention the sensor for a marker gas is a sensor for SF₆, methane or hydrogen gas.

In a particularly preferred device according to the invention the sensor for a marker gas is a sensor for hydrogen gas.

Emphasis is given to a device for monitoring the functioning of the lower esophageal sphincter in mammals which comprises a measuring probe, a sensor for hydrogen gas and a device which records the amount of hydrogen gas detected in the sensor dependent of time.

Particular mention (aspect A1) may be made of a device for monitoring the functioning of the lower esophageal sphincter in mammals which comprises a measuring probe head, a sensor for a marker gas, a first tube adducting transporting gas to the measuring probe head, a second tube leading the mixture of the transporting gas and the marker gas which is collected from the environment of the measuring probe head to the sensor for the marker gas, a vacuum pump which sucks the mixture of the transporting gas and the marker gas which is collected from the environment of the measuring probe head to the sensor for the marker gas, and a device which records the amount of marker gas detected in the sensor dependent of time.

Preferred (aspect A2) is a device according to aspect A1 wherein the sensor for a marker gas is a semiconductor sensor for the marker gas.

Particularly preferred (aspect A3) is a device according to aspect A2 or aspect A3 wherein the sensor for a marker gas is a sensor for hydrogen gas.

Emphasis (aspect A4) is therefore given to a device for monitoring the functioning of the lower esophageal sphincter in mammals which comprises a measuring probe head, a semiconductor sensor for hydrogen gas, a first tube adducting transporting gas to the measuring probe head, a second tube leading the mixture of the transporting gas and hydrogen gas which was collected from the environment of the measuring probe head to the semiconductor sensor for hydrogen gas, a vacuum pump which sucks the mixture of the transporting gas and hydrogen gas which was collected from the environment of the measuring probe head to the semiconductor sensor for hydrogen gas, and a device which records the amount of hydrogen gas detected in the sensor dependent of time.

Another aspect of the invention (aspect B) relates to the use of a sensor for a marker gas in a device for monitoring the functioning of the lower esophageal sphincter in mammals.

Preferred is the use of a sensor for hydrogen gas in a device for monitoring the functioning of the lower esophageal sphincter in mammals.

Another aspect of the invention (aspect C) relates to the use of a measuring probe comprising a dosed measuring probe head in a device for monitoring the functioning of the lower esophageal sphincter in mammals.

Preferred is the use of a measuring probe comprising a closed measuring probe head in a device for monitoring the functioning of the lower esophageal sphincter in mammals, whereby the inside of the measuring probe head is separated from the environment of the measuring probe by a membrane.

Particularly preferred is the use of a measuring probe comprising a closed measuring probe head in a device for monitoring the functioning of the lower esophageal sphincter in mammals, whereby the inside of the measuring probe head is separated from the environment of the measuring probe by a membrane, which is preferably or selectively penetrable for a marker gas.

Particularly emphasis is given to the use of a device according to aspect A, A1, A2, A3 or A4 for monitoring the functioning of the lower esophageal sphincter in mammals.

Still another aspect of the invention (aspect D) relates to a method for monitoring the functioning of the lower esophageal sphincter in mammals which method comprises the monitoring of a marker gas which refluxed from the stomach into the esophagus.

The invention further relates to a method for monitoring the functioning of the lower esophageal sphincter in mammals which method comprises the monitoring of a marker gas which refluxed from the stomach into the esophagus, wherein the marker gas which refluxed from the stomach into the esophagus is monitored continuously.

The invention further relates to a method according to the invention, wherein the marker gas is continuously introduced into and/or evolved in the stomach.

In a preferred method according to the invention, the marker gas is selected from the group consisting of SF₆, methane and hydrogen.

In a particularly preferred method according to the invention, the marker gas is hydrogen.

In a preferred method according to the invention, the measuring probe is placed in the esophagus.

Emphasis is given to a method for monitoring the functioning of the lower esophageal sphincter in humans which comprises the monitoring of hydrogen which refluxed from the stomach into the esophagus and wherein the measuring probe is placed in the esophagus.

Particular emphasis is given to a method for monitoring the functioning of the lower esophageal sphincter in humans which comprises the monitoring of hydrogen which refluxed from the stomach into the esophagus and wherein the measuring probe is placed in the esophagus and the hydrogen is evolved continuously in the stomach.

Particular emphasis is also given to a method for monitoring the functioning of the lower esophageal sphincter in warm blooded animals which comprises the monitoring of hydrogen which refluxed from the stomach into the esophagus and wherein the hydrogen is pumped into the stomach.

Still another aspect of the invention (aspect E) relates to the use of a sensor for a marker gas for the production of a device for monitoring the functioning of the lower esophageal sphincter in mammals.

The invention further relates to the use of a sensor for hydrogen gas for the production of a device for monitoring the functioning of the lower esophageal sphincter in mammals.

The device and method of monitoring the functioning of the lower esophageal sphincter by monitoring a marker gas which refluxed from the stomach into the esophagus are useful in the diagnosis of dysfunctions of the lower esophageal sphincter in mammals. More specifically the device and the method can be used inter alia in the diagnosis of GERD.

One advantage of the method and the device according to the invention is that the monitoring of the functioning of the lower esophageal sphincter can be performed irrespective of the pH value of the gastric contents and irrespective of the pH value of the reflux. Therefore the method according to the invention is for example also applicable to individuals who are treated with pharmaceuticals which inhibit acid secretion, such as for example histamine H₂-blockers (e.g. cimetidine, ranitidine, famotidine) or H⁺/K⁺-ATPase inhibitors (e.g. omeprazole, esomeprazole, lansoprazole, pantoprazole or rabeprazole), acid pump antagonists or potassium competitive acid blockers (p-CABs).

A further advantage of the method and the device according to the invention is that the monitoring of the functioning of the LES can be performed irrespective of the state of aggregation of the reflux, because the marker gas can get into contact with and penetrate the measuring probe either as a gas or dissolved in liquid reflux. Thus, this technique measures refluxes independent of the pH and of the type of reflux and therefore refluxes can be detected with this method according to the invention which might be not detectable by the other techniques known in the art.

Still a further advantage of the method and the device according to the invention is an advantageous time-based resolution in the detection of TLESR which allows inter alia the monitoring of two TLESRs which occur within a short period of time, for example two successive TLESR-induced refluxes which occur within a time period of 10 seconds or even less, for example between 1 and 10 seconds. In contrast to the pH-metry technique, where buffering effects affect the time resolution of the registration and peristaltic waves have to occur to empty the esophagus from the refluxed acid, the retention period of hydrogen is very short and therefore short successions of reflux events can be identified.

Still a further advantage of the method and the device according to the invention is that the device and especially the measuring probe, which is to be used for this method, can be miniaturized to a size and a thickness which allows an easy application. If the measuring probe is to be placed in the esophagus and therefore has to be swallowed by the individual, a miniaturized size will be of advantage with regard to ease of swallowing and therefore to acceptance of the technique in general by the patients being under observation.

Still a further advantage of the method according to the invention is that this method is applicable not only in clinical but also in ambulatory use due to its simple technical equipment.

### DETAILED DESCRIPTION OF THE INVENTION

In the following, the various aspects of the present invention are described in more detail.

Preferred embodiments of the present invention are subject of the dependent claims.

The term mammals according to the invention includes warm blooded animals such as dogs, cats, rats, opossums, mice, pigs and the like, as well as humans.

Marker gases according to the invention are gases which can be introduced into or evolved in the stomach of a mammal in concentrations needed for the detection of said marker gas without causing serious side effects and which can be detected, preferably selectively, by a suitable sensor. The marker gas also can be mixed, for example prior to the introduction into the stomach, with a carrier gas, like for example nitrogen (N₂) or air, in which the marker gas is diluted and which carrier gas itself is not detected by the sensor for the marker gas. Examples of suitable marker gases according to the invention are inter alia SF₆, methane and especially hydrogen (H₂).

The concentration of the marker gas in the mixture with any other gas for detection of the marker gas in the sensor for the marker gas according to the invention can range from 0.0001-100%, preferably from 0.001-100%. The minimum concentration needed for appropriate detection depends on the nature of the marker gas and on the sensitivity of the sensor for that marker gas. As an example, usual sensitivity of commercially available sensors for hydrogen limits usage of hydrogen concentrations of about 0.0001% in a mixture with the any other gas according to the invention. Highest sensitivity in that case can be achieved using a mixture of gases comprising 3.5% of hydrogen. Higher concentrations of hydrogen as the marker gas can only be used under highly controlled conditions.

The introduction of the marker gas into the stomach can be achieved for example by continuously or discontinuously pumping the marker gas, preferably in a mixture with a suitable carrier gas, into the stomach. Hereby, the introduction of the marker gas into the stomach can be achieved via different pathways, like for example via a fistula, which has to be inserted to the stomach by an operation known to the expert as an additional entry/exit of the stomach. This pathway of introduction of the marker gas into the stomach via a fistula is, however, not applicable for standard diagnosis especially in humans, but, however, particularly suitable for the establishment and improvement of the device and the method according to the invention in animal studies. As an alternative, the introduction of the marker gas into the stomach can be achieved via a tube, cannula or other means known to the expert, which was introduced into the esophagus via the mouth or the nose, swallowed and introduced into the stomach by passage through the LES. The diameter of the tube or the cannula, via which the marker gas is to be pumped into the stomach, however has to be thin enough in order not to disturb the natural functioning of the LES. Another alternative is the operation of an esophagostomy, a mucosa to skin anastomosis, yielding the admission to the lower esophagus in animal investigations.

In order to maintain a defined pressure difference between the stomach and the esophagus, which is needed in order to provoke reproducible TLESRs, the marker gas is pumped into the stomach by use of a barostat. The barostats which can be used for the present invention, are known to a person skilled in the art. Such barostats have been used in the prior in an analogous manner in animal models in order to upbuild and maintain a overpressure in the stomach, for example in order to study accommodation of the stomach to an overpressure. For the present invention suitable pressure differences between the stomach and the esophagus (barrier pressure) are between 0.5 and 30 mm Hg, preferably between 2 and 20 mmHg, especially between 8 and 18 mm Hg.

Alternatively, instead of pumping into the stomach, the marker gas can be introduced into the stomach by evolution or liberation of the marker gas directly in the stomach, for example by chemical and/or microbiological production.

Alternatively and with particular emphasis, the marker gas to be monitored, especially in the case of hydrogen, is evolved naturally in the stomach without additional and/or artificial chemical and/or microbiological production. Digestive enzymes in the stomach and upper small intestine digest carbohydrates in the food we eat and bacteria in the lower small intestine digest the rest of the carbohydrates, and produce hydrogen gas. This natural evolution of hydrogen gas can only be monitored by a device according to the present invention, if the hydrogen sensitivity of the device according to the present invention is between 0.1 and 200, in particular between 2 and 50 ppm. This sensitivity is achieved by a device according to the invention. The natural production of hydrogen gas can be enhanced, if desired, for example by additional digestion of lactulose.

The device and the method according to the present invention therefore is useful for application to individuals in their natural condition without artificial influence on the contents of the stomach.

Furthermore, the device and the method according to the present invention can either be applied to individuals, where TLESRs are provoked by the introduction and/or evolution of gas in the stomach or to individuals, where TLESRs are not provoked in such a way. The latter is particularly preferred for application of the device and the method according to the present invention in humans.

In the case of hydrogen, which is the most preferred marker gas according to the invention, the optional microbiological production of hydrogen can be achieved for example by microorganisms being completely applicable to mammals (for example E. coli Nissle 1917). These microorganisms can be administered to the mammal by methods known to the expert, for example in a capsule together with a sufficient amount of a suitable substrate, like for example glucose. Due to the fact that the intragastric oxygen concentration is low and a considerable amount of bacteria is incorporated in the capsule, the bacteria will metabolize the substrate mostly by anaerobic pathways, leading to the production of hydrogen. Depending on the composition and the content of the capsule, the hydrogen production proceeds continuously during the experimental run.

The term “monitoring of a marker gas which refluxed from the stomach into the esophagus” is to be understood as monitoring and recording of the time dependent concentration of the marker gas present in the gas that is analyzed in the sensor of the marker gas. The gas that is analyzed in the sensor of the marker gas is the gas which is sucked or absorbed into the measuring probe and is mixed, if desired or technically necessary, with an additional transporting gas, such as nitrogen (N₂) or air, whereby the transporting gas supports the transport of the marker gas to the sensor of the marker gas. If a transporting gas is to be used, the measuring probe is suitably composed of 2 thin tubes, one adducting the marker gas free transporting gas and the second leading away the mixture of the transporting gas and the marker gas which was collected from the environment of the measuring probe head.

Measuring probes according to the invention are devices which collect the gas comprising the marker gas. The collection of gas by the measuring probe takes place in the measuring probe head and is performed by methods known to the expert, for example by suction. One embodiment (embodiment a) of a measuring probe comprises an open measuring probe head whereby the gas around the measuring probe containing the marker gas enters the measuring probe head without passing a barrier. An example of an open measuring probe head is an open tube. Another embodiment (embodiment b) of a measuring probe comprises a dosed measuring probe head, whereby the inside of the measuring probe head is separated from the environment of the measuring probe by a membrane, for example by a foil. The marker gas has to penetrate the membrane in order to be collected in the measuring probe head. Preferably the membrane is more penetrable for the marker gas as compared to the other components of the gas in the environment of the measuring probe. Especially preferred are membranes which are preferably or selectively penetrable for the marker gas, so that from the gas in the environment of the measuring probe mainly or only the marker gas penetrates the membrane to the inside of the measuring probe head. The function of the membrane thus can be compared to a filter which can mainly or only be passed by the marker gas. Such a marker gas selectivity of the membrane results in an undisturbed registration of the marker gas in the sensor.

The measuring probe head is connected to the sensor for the marker gas in manner known to a person skilled in the art in order to assure transportation of the marker gas to the sensor, for example via a tube. The gas comprising the marker gas is lead away from the measuring probe head through this tube to the sensor of the marker gas. For miniaturization of the measuring probe comprising the measuring probe head and the tubes, these tubes can be capillary tubes of a diameter between 0.05 and 5 mm, preferably between 0.2 and 2 mm, especially between 0.5 and 1 mm.

The transportation of the gas collected in the measuring probe head to the sensor for the marker gas is performed in a manner which is familiar to a person skilled in the art, for example by suction and/or transportation using a transporting gas, like for example nitrogen (N₂) or air.

In a preferred embodiment of the invention two tubes are connected to the measuring probe head. The first tube is adducting the transporting gas to the measuring probe head. In the measuring probe head the gas which is collected form the environment of the measuring probe head is admixed with the transporting gas and the mixture is further lead through the second tube to the sensor of the marker gas. Particularly preferred is such an embodiment where a vacuum pump sucks the mixture of the transporting gas and the marker gas which is collected from the environment of the measuring probe head to the sensor for the marker gas. This constant carry away of the marker gas keeps the marker gas gradient across the probe head membrane steep and therefore warrants the fast transportation of the marker gas across the membrane of the probe head to the sensor and, if a closed measuring probe head is used, a fast diffusion of the marker gas through the membrane. This in turn leads to a high time resolution in the detection of the marker gas.

The devices of the present invention where the measuring probe is placed in the esophagus as described above can additionally be combined with an impedance electrode, which is in principle known to a person skilled in the art. This combination leads to a more reliable differentiation between solid and gaseous gastroesophageal refluxes thus broadening the amount of information obtained by the method according to the present invention for each reflux event. The additional implementation of such an electrode will not make the measuring probe head significantly thicker and therefore will not have significant influence on patient compliance. Such a combination with a impedance electrode is a particularly preferred embodiment of the present invention.

The invention therefore also relates to a device according to the present invention for monitoring the functioning of the lower esophageal sphincter in mammals wherein the device comprises a device as described above for monitoring of a marker gas which refluxed from the stomach into the esophagus and additionally an impedance electrode.

For the collection of the gas comprising the marker gas, the measuring probe can be placed outside of the body where the gas, which refluxed from the stomach into the esophagus, leaves the body, especially in front of the mouth and/or of the nose. Alternatively, the measuring probe can be introduced, for example by swallowing, into the esophagus of the mammal. In this case, the measuring probe is preferably placed above the LES, especially in a distance above the LES where the presence of the measuring probe has no effect on the functioning of the LES. Methods which allow to place a measuring probe in a suitable distance above the LES are known to a person skilled in the art, for example from methods using pH or impedance probes. As an example the probe is introduced into the esophagus until a pressure sensor indicates the passage through the LES. After the indication of the passage through the LES, the probe is pulled out until the desired distance above the LES is reached. The measuring probe is preferably placed in the esophagus between 2 and 10 cm, especially between 4 and 6 cm above the LES.

Sensors for the marker gas according to the invention are devices which detect the concentration of the marker gas in a mixture of gases time dependently. Sensors for the marker gases which can be used in the present invention, are commercially available, like for example semiconductor gas sensors (for example for hydrogen: or SF₆ and the like, sensor systems also commercially available) or infrared gas sensors (inter alia for methane). Semiconductor sensors are the sensors preferably used in a device according to the invention, espedally those semiconductor sensors which are highly and fast responding due to superficial absorption (not diffusion into the semiconductor) of the marker gas that modifies the conductance. The sensor for the marker gas is connected to the measuring probe via a suitable connection which connections are known to a person skilled in the art. The transportation of the gas collected in the measuring probe to the sensor for the marker gas is also performed in a manner which is familiar to a person skilled in the art, for example by suction or transportation using a transporting gas, like for example nitrogen (N₂) or air.

The device which records the amount of marker gas detected in the sensor time dependently is a device where the concentration of the marker gas in the gas analyzing sensor is recorded continuously during the time of the measurement. This device therefore stores individual concentrations of the marker gas in the gas analyzed in the sensor as a function of time. Significant enhancement above the base line values in the concentration of the marker gas in the analyzed gas can be defined to represent a reflux from the stomach into the esophagus. Significant enhancement means an enhancement of the marker gas concentration being significantly different from the scattering of the base line. As an example of a significant enhancement, the occurrence of a peak in a concentration of the marker gas versus time plot is to be mentioned, which can be associated with a transient lower esophageal sphincter relaxation.

The functioning of the lower esophageal sphincter in terms of the present invention is characterized inter alia by the frequency of the occurrence of transient lower esophageal sphincter relaxations (number of TLESRs in a defined time period), slope of onset and of offset, level and duration of the level between two peaks in a plot of concentration of the marker gas detected in the sensor versus time. Moreover, the peak broadness gives informations on the duration of TLESR. The height of the amplitude gives some additional information on the concentration of the marker gas in refluxed gas or fluids. Monitoring the functioning of the lower esophageal sphincter therefore is to be understood as the detection, for example of the number, the duration, the extent and further characteristics of TLESR induced refluxes which occurred under defined conditions during a certain time period.

Determination of changes in function of the LES can typically be done by measuring the rate of TLESRs upon administration of a compound that inhibits the occurance of TLESRs and to compare it with the compound free control. This procedure can be used inter alia to establish dose-activity relationships for different compounds inhibiting the occurrence of TLESRs.

### MODE(S) FOR CARRYING OUT THE INVENTION

The following examples serve to illustrate the invention in more detail without restricting it. In particular the invention is not restricted to be applicable to the species mentioned in the examples. Further species such as for example rat, mouse (for example gerbil), guinea pig, opossum (for example monodelphis domestica) or other animals can be tested using technical and methodological details which are identical or similar to those described in the examples and which can be adopted by a person skilled in the art. In particular for application in humans, however, the introduction of the marker gas into the stomach has to be solved differently as in the examples, for example as described in more detail in the description of the invention. Alternatively, the natural evolution of hydrogen gas in the stomach of mammals, in particular humans, can be used for the detection of TLESR induced refluxes which are not provoked by the introduction and/or evolution of gas in the stomach of the individuals.

Furthermore the invention is not restricted to the devices and methods used in the examples. Further devices and methods which work in an analogous manner, for example as described in more detail in the description of the invention, or in a manner known to the person skilled in the art, can likewise be used.

Dog with a Gastric Fistula

The various aspects of the invention were studied with fistula dogs. The fistula is inserted into the stomach of the dog by methods known to the expert, for example as described in Postius S, Braeuer U. Loss of circadian rhythm in luminal acidity of canine stomach by implantation of a gastric fistula. Digestion. 1995; 56(1):3540.

The method according to the invention was used to quantify the number of transient lower esophageal sphincter relaxations (TLESRs, leading to gas refluxes) in the conscious dog, which are provoked by the introduction of hydrogen gas in the stomach. The method can be used with fasted or fed animals and it is not depending on the status of gastric acid secretion.

For the assessment of TLESRs, the gastric fistula dogs were temporarily connected via the gastric fistula to a barostat which continuously measures the gastric pressure and continuously approximates during the monitoring of the marker gas a target pressure by sucking or pumping a gas mixture of air and hydrogen, comprising 0.2-3% hydrogen, whereby 3% of hydrogen was yielding the highest sensitivity.

A target pressure was selected that causes an appropriate number of TLESRs, usually for a period of 30 min. Appropriate means that there has to be a sufficiently high number of TLESRs to enable estimation of a compound-induced reduction of the number of TLESRs, but, on the other hand, not too many, since the registration technique with the equipment used had a good resolution of about 10-15 eructations/minute. The quantification of TLESRs resulting in eructations was performed by continuous collection of the gas in front of and in the middle of nose and mouth of the dog with an open measuring probe head. This gas, comprising hydrogen from the gastric gas mixture, which eructed if the dog is belching, was sucked to a hydrogen sensor (Model used: UST 2330, UST—Umweltsensortechnik GmbH, 98716 Geschwenda, Germany) registering the hydrogen concentration. Significant enhancement in hydrogen concentration in the collected gas was defined to represent a TLESR caused by an eruction. Significant means an enhancement being significantly different from the scattering of the base line. No eructations were usually caused in this model by swallows nor do TLESR/eructations occur without elevated gastric pressure. The threshold for the induction of eructation has been found to be between 8 and 10 mm Hg. Some eructions occurred at 6 mm Hg and lots were measured above 12 mm Hg. However, above 12-14 mm Hg vomiting might be induced as well.

The results obtained in this newly developed test system clearly demonstrate the potential of this in vivo model with respect to an easy, fast and reliable assessment for compounds that inhibit TLESRs.

The following table 1 demonstrates the differences measured using a device and method according to the invention under the conditions mentioned above between animals to which were administered compounds inhibiting the number of TLESRs (drug group) and animals to which these compounds were not administered (control group). The drug group received Baclofen, a GABA B agonist, in a dose of 7 μmol/kg i.v., which is known to strongly inhibit the number of TLESRs. The study design was as follows:

30 minutes: administration of 7 μmol/kg i.v. Baclofen or placebo

10 minutes: dog connected pressure free to the barostat,

0 minutes onset of 12 mm Hg to the stomach,

0-30 minutes: detection of the number of TLESR by monitoring the concentration of hydrogen detected in the sensor

30 min end of experimental run.

TABLE 1

| | Number of TLESR detected in | | |
|---|---|---|---|
| | the time period indicated | | |
| | Time period | | |
| | 0-10 minutes | 10-20 minutes | 20-30 minutes |
| Control group | 13.00 | 4.25 | 2.00 |
| Baclofen group | 0.75 | 0.25 | 1.75 |

Human

The various aspects of the invention can further be studied with humans where, for example, the monitoring of naturally evolved hydrogen gas in human stomachs is used for the detection of TLESR induced refluxes.

The method and the device according to the invention can be used to quantify the number of transient lower esophageal sphincter relaxations (TLESRs, leading to gas and/or liquid refluxes) in conscious mammals, whereby the TLESRs are neither provoked by either introduction of gas or artificial production gas, intake of liquid volume nor by intake of food. The method can be used with warm blooded animals or humans and it is not depending on the status of gastric acid secretion thus enabling the natural (physiological or pathophysiological) pattern of gastroesophageal refluxes.

A closed measuring probe head (preferably cylindrical in shape with a diameter of about 1-3 mm and a length of about 0.5-4 mm) can be introduced by swallowing into the esophagus of the human and can be placed about 5 cm above the LES. The closed measuring probe head comprises a membrane which is permeable for hydrogen gas only or for gases in general but excluding permeation of water. The membrane can be for example either a metal foil such as niobium or other hydrogen-permissive metal foils and alloys thereof, or a foil of another material known to be permeable for gases. This technique warrants the transport of diffusing hydrogen gas to the sensor to be measured in an undisturbed manner. The foil or membrane mainly has to avoid the entering of humidity into the gas flow onto the sensor.

The measuring probe head is connected to two parallel capillary tubes (each of a diameter of about 0.2-2 mm, preferably being melt or glued together to yield one dual channeled tube), one of these capillary tubes as inlet for the transporting gas, i.e. air or other suitable gases or mixture of gases and the second tube (or channel) adducting the mixture of the transporting gas and the hydrogen gas which is collected from the environment of the measuring probe head to the sensor for hydrogen gas registering the hydrogen concentration.

The flow of the gas is managed by a pump before or after the sensor for the hydrogen gas, creating a moderate vacuum, for example of 1-10 mmHg, thus sucking the gas mixture out of the system.

The device of the present invention as described for application in humans can additionally be combined with an impedance electrode. This combination leads to a reliable differentiation between solid and gaseous gastroesophageal refluxes. This technique is well known for a skilled person in this field. The implementation of such an electrode is not expected to make the double channeled tube or the measuring probe head much thicker and therefore will not have significant influence on patient compliance.

1. A device for monitoring lower esophageal sphincter function in mammals which comprises a sensor for a marker gas.

2. A device for monitoring lower esophageal sphincter function in mammals which comprises a measuring probe, a sensor for a marker gas and a device which records the amount of marker gas detected in the sensor dependent of time.

3. A device as claimed in claim 1, wherein the sensor for a marker gas is a sensor for SF₆, methane or hydrogen gas.

4. A device as claimed in claim 1, wherein the sensor for a marker gas is a sensor for hydrogen gas.

5. A device for monitoring lower esophageal sphincter function in mammals which comprises a measuring probe, a sensor for hydrogen gas and a device which records the amount of hydrogen gas detected in the sensor dependent of time.

6. A device for monitoring lower esophageal sphincter function in mammals which comprises
a measuring probe head,
a sensor for a marker gas,
a first tube adducting transporting gas to the measuring probe head,
a second tube leading a mixture of the transporting gas and the marker gas which is collected from an environment of the measuring probe head to the sensor for the marker gas,
a vacuum pump which sucks the mixture of the transporting gas and the marker gas which is collected from the environment of the measuring probe head to the sensor for the marker gas,
and a device which records the amount of marker gas detected in the sensor dependent of time.

7. A device as claimed in claim 6 wherein the sensor for a marker gas is a semiconductor sensor for the marker gas.

8. A device as claimed in claim 6 wherein the sensor for a marker gas is a sensor for hydrogen gas.

9. A device for monitoring lower esophageal sphincter function in mammals which comprises
a measuring probe head,
a semiconductor sensor for hydrogen gas,
a first tube adducting transporting gas to the measuring probe head,
a second tube leading a mixture of the transporting gas and hydrogen gas which was collected from an environment of the measuring probe head to the semiconductor sensor for hydrogen gas,
a vacuum pump which sucks the mixture of the transporting gas and hydrogen gas which was collected from the environment of the measuring probe head to the semiconductor sensor for hydrogen gas,
and a device which records the amount of hydrogen gas detected in the sensor dependent of time.

10. A method for monitoring lower esophageal sphincter function in mammals, comprising introducing a marker gas into the mammal's stomach and monitoring the marker gas with a device according to claim 1.

11. The method for monitoring lower esophageal sphincter function in mammals according to claim 10, wherein the marker gas is hydrogen.

12. (canceled)

13. (canceled)

14. (canceled)

15. (canceled)

16. (canceled)

17. A method for monitoring lower esophageal sphincter function in mammals which method comprises monitoring of a marker gas which is refluxed from the mammal's stomach into the esophagus.

18. The method according to claim 17, wherein the marker gas is selected from the group consisting of SF6, methane and hydrogen.

19. The method according claim 17, wherein the marker gas is hydrogen.

20. A method for monitoring lower esophageal sphincter function in humans which comprises monitoring of hydrogen which is refluxed from the human's stomach into the human's esophagus with a measuring probe, wherein the measuring probe is placed in the esophagus.

21. A method for monitoring lower esophageal sphincter function in humans which comprises monitoring of hydrogen which is refluxed from the human's stomach into the human's esophagus with a measuring probe, wherein the measuring probe is placed in the esophagus and the hydrogen is evolved continuously in the stomach.

22. A method for monitoring lower esophageal sphincter function in warm blooded animals which comprises monitoring of hydrogen which is refluxed from the warm blooded animal's stomach into the warm blooded animal's esophagus and wherein the hydrogen is pumped into the stomach.
